(19) United States
(12) Patent Application Publication
(10) Pub. No.: US 2014/0345648 A1
(43) Pub. Date: Nov. 27, 2014

(54) Oral Healthcare, Products and Methods Therefor

(71) Applicant: Hisham Abdalla, Auckland (NZ)

(72) Inventor: Hisham Abdalla, Auckland (NZ)

(73) Assignee: OZOSPA HOLDINGS LIMITED, Auckland (NZ)

(21) Appl. No.: 14/365,247

(22) PCT Filed: Dec. 15, 2011
(86) PCT No.: PCT/NZ11/00263

§ 371 Date: Aug. 4, 2014

(51) Int. Cl. A61K 33/20; A61K 31/047; A61K 31/661; A61Q 11/00

(52) U.S. Cl. CPC A61K 33/20; A61Q 11/00; A61K 31/047; A61K 31/661; A61K 2800/74
USPC 134/7; 424/49

(57) ABSTRACT

The present invention relates to oral health. In particular, though not solely, the present invention is directed to products and methods for use by a consumer on a regular basis to maintain and improve their oral health as part of their oral health care program.

### TECHNICAL FIELD OF THE INVENTION

The present invention relates to oral health. In particular, though not solely, the present invention is directed to products and methods for use by a consumer on a regular basis to maintain and improve their oral health as part of their oral health care program.

### BACKGROUND OF THE INVENTION

Tooth decay and gum disease are prevalent worldwide. The financial, health, psychological and social impacts of these issues are of concern to individuals, governments and international organisations such as WHO (World Health organisation).

Dental and oral care products currently on the market that claim to treat or prevent tooth decay and gum disease often contain ingredients which can have negative side effects. For example many products contain propylene glycol which is a potential oral mucosa irritant. Many products also contain alcohols and sodium lauryl sulphate which are desiccating agents and known irritants. Ingredients which are desiccating dry out the mouth and reduce the naturally beneficial effect of saliva, especially in individuals with impaired saliva production or poor saliva quality (for example xerostomics, elderly, heavily medicated individuals, smokers, those with diets high sugar, high in caffeine, high in alcohol and/or high in acidic drinks). Some oral care products can make the naturally neutral pH environment in the mouth acidic, which can cause demineralisation and erosion. In addition, some ingredients which are included as a means to thoroughly clean the teeth, (for example silica) can be too abrasive, resulting in the wearing away of the protective enamel on the teeth after prolonged use.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

It is an object of the present invention to provide improved oral health care products and methods, or to overcome the above shortcomings or address the above desiderata, or to at least provide the public with a useful choice.

### BRIEF DESCRIPTION OF THE INVENTION

In a first aspect the present invention may be said to broadly consist in a method of improving the health of the oral cavity, comprising or including the steps in any order of:
a) introducing into said oral cavity a liquid product containing at least:
   about 5% to about 50% w/v of a saliva activating agent, an oxidising agent, and water, thereafter removing said liquid product at least in part from the oral cavity,
b) introducing into said oral cavity, a product at least initially in powder form
   (“powder product”) containing at least the following ingredients,
      about 30% to about 98% w/w of a saliva activating agent,
      a pH adjusting agent, and
      a remineralising agent,
thereafter agitating said powder product, whether dissolved or otherwise, in said oral cavity at least in part with said oral tool, and thereafter removing the resultant product or products at least in part from the oral cavity.

Preferably said saliva activating agent in said liquid product is present in a range of about 5% to about 30% w/v.

Preferably said saliva activating agent in said liquid product is present in a range of about 5% to about 15% w/v.

Preferably said saliva activating agent in said liquid product is present at about 10% w/v.

Preferably said saliva activating agent in said liquid product is non-acidic.

Preferably said saliva activating agent in said liquid product is non-fermentable

Preferably said saliva activating agent in said liquid product is a caries reducing agent.

Preferably said saliva activating agent in said liquid product is a plaque adhesion reducing agent.

Preferably said saliva activating agent in said liquid product is xylitol.

Preferably said oxidising agent in said liquid product is present in a range of about 200 PPM to about 1000 PPM in solution.

Preferably said oxidising agent in said liquid product is present in a range of about 300 PPM to about 700 PPM in solution.

Preferably said oxidising agent in said liquid product is present at about 500 PPM in solution.

Preferably said oxidising agent has a whitening and/or bleaching effect on teeth in said oral cavity.

Preferably said oxidising agent at least temporarily reduces or temporarily substantially alleviates anaerobic bacteria in said oral cavity.

Preferably said oxidising agent at least partially oxidises volatile sulphur compounds in said oral cavity, reducing instance of malodorous breath.

Preferably said oxidising agent is selected from anyone or more of the following:
stabilised chlorine dioxide,
hydrogen peroxide,
zinc chloride.

Preferably said oxidising agent is stabilised chlorine dioxide.

Preferably said liquid product contains water in a range of about 80% to about 90% by weight.

Preferably said liquid product contains a non-fermentable flavouring agent.

Preferably said non-fermentable flavouring agent is menthol.

Preferably said liquid product also contains any one or more of the following:
a remineralising agent,
a stabilising agent,
a preservative.

Preferably said liquid product has a pH of about 7 to about 9.

Preferably said liquid product has a pH of about 7.5 to about 8.8

Preferably said liquid product has a pH of about 7.6 to about 8.5.

Preferably said liquid product is substantially non-coloured.

Preferably said liquid product is non-desiccating when introduced into said oral cavity.

Preferably said saliva activating agent in said powder product is present in a range of about 30% to about 80% w/w.

Preferably said saliva activating agent in said powder product is present in a range of about 40% to about 70% w/w.

Preferably said saliva activating agent in said powder product is present in a range of about 40% to about 60% w/w.

Preferably said saliva activating agent in said powder product is present at about 50% w/w.

Preferably said saliva activating agent in said powder product is non-acidic.

Preferably said saliva activating agent in said powder product is non-fermentable.

Preferably said saliva activating agent in said powder product is a caries reducing agent.

Preferably said saliva activating agent in said powder product is a plaque adhesion reducing agent.

Preferably said saliva activating agent in said powder product is xylitol.

Preferably said pH adjusting agent in said powder product is present in a range of about 1% to about 50% by weight.

Preferably said pH adjusting agent in said powder product is present in a range of about 20% to about 50% by weight.

Preferably said pH adjusting agent in said powder product is present is present in a range of about 30% to about 40% by weight.

Preferably said pH adjusting agent in said powder product is present at about 40% by weight.

Preferably said pH adjusting agent is sodium bicarbonate.

Preferably said remineralising agent in said powder product is present in a range of about 0.1% to about 30% by weight.

Preferably said remineralising agent in said powder product is present in a range of about 5% to about 20% by weight.

Preferably said remineralising agent in said powder product is present at about 10% by weight.

Preferably said remineralising agent in said powder product provides soluble mineral ions.

Preferably said remineralising agent is selected from any one or more of the following:
calcium glycerophosphate,
sodium fluoride.

Preferably said remineralising agent is calcium glycerophosphate

Preferably said powder product has a pH of about 7 to about 9.

Preferably said powder product has a pH of about 7.8 to about 8.2.

Preferably said powder product has a pH of about 8.

Preferably said powder product substantially dissolves when introduced into said oral cavity.

Preferably said powder product is substantially non-abrasive when introduced into said oral cavity.

Preferably said powder product contains a non-fermentable flavouring.

Preferably said non-fermentable flavouring is menthol.

Preferably said powder product also contains any one or more of the following:
a stabilising agent,
a preservative,
a filler.

Preferably said saliva activating agent is the same in said liquid product and said powder product.

Preferably said saliva activating agent is xylitol in both said liquid product and said powder product.

Preferably said liquid product is retained in the oral cavity for about 10 seconds to about 2 minutes.

Preferably said liquid product is retained in the oral cavity for about 1 minute.

Preferably said liquid product is removed from the oral cavity by expectorating.

Preferably said powder product is removed from the oral cavity by expectorating.

Preferably said powder product is introduced into the oral cavity, and/or is agitated in the oral cavity with an oral tool, that is preferably selected from any one or more of the following:
a dental brush,
floss,
gauze,
finger dental brush.

Preferably said agitation is over any one or more of the following,
teeth,
gums,
tongue, and
cheek(s).

Preferably said agitation is at least over said teeth.

Preferably the steps in the method are carried out in the order of step a) followed by step b).

Preferably said liquid product in step a) neutralises acid in said oral cavity prior to carrying out step b).

Preferably said method includes repeating steps a) and b) on at least a daily basis.

Preferably said method includes repeating steps a) and b) at least twice in a 24 hour period, for example, in the morning and in the evening.

Preferably said method includes, in addition to and after steps a) and b) introducing into said oral cavity a lozenge product containing at least the following ingredients,
about 30% to about 95% by weight of a saliva activating agent, and a remineralising agent.

Preferably said lozenge product contains a pH adjusting agent.

Preferably said lozenge product contains a non-fermentable flavouring agent.

Preferably said non-fermentable flavouring agent is selected from any one or more of the following:
liquorice extract,
menthol,
natural oil.

Preferably said lozenge product contains at least one binding agent.

Preferably said binding agent is selected from any one or more of the following:
maltitol,
mannitol,
arabic gum.

Preferably said lozenge product is selected from any one or more of the following:
a dissolvable lozenge,
a chewable tablet, whether gum based or otherwise.

Preferably said lozenge product is introduced into said oral cavity at least once in a 24 hour period.

Preferably said lozenge product is introduced into said oral cavity at least three times in a 24 hour period.

Preferably said lozenge product is introduced into said oral cavity within about 30 minutes of eating or drinking.

Preferably said lozenge product is introduced into said oral cavity within about 10 minutes of eating or drinking.

In another aspect the present invention provides in a product for improving the health of the oral cavity, the product comprising or including,
about 30% to about 98% w/w of a saliva activating agent,
a pH adjusting agent,
an oxidising agent, and
a remineralising agent,
   wherein said saliva activating agent activates stimulated saliva in said oral cavity, said pH adjusting agent adjusts and buffers the pH of said oral cavity to pH range of about 6.8 to about 7.2, and said remineralising agent provides an excess of at least one mineral to at least reduce demineralisation of at least one tooth in said oral cavity.

Preferably said product is any one or more of the following:
a powder product,
a liquid product,
a lozenge or tablet (whether gum-based or otherwise),
a floss
oral spray.

In another aspect the present invention provides in a product for improving the health of the oral cavity, comprising or including,
about 30% to about 98% w/w of a saliva activating agent,
a pH adjusting agent, and
a remineralising agent,
   wherein said product has a pH of about 7.8 to about 8.2, and
   wherein said product is in solid form and wherein said product substantially dissolves when introduced into said oral cavity.

Preferably said powder product is substantially non-abrasive when introduced into said oral cavity.

In yet another aspect the present invention consists in a kit of parts for improving the health of the oral cavity, comprising or including a powder product and a liquid product, wherein said liquid product contains at least:
about 5% to about 50% w/v of a saliva activating agent,
an oxidising agent, and
water,
   and said powder product contains at least:
about 30% to about 98% w/w of a saliva activating agent,
a pH adjusting agent, and
a remineralising agent.

Preferably said saliva activating agent in said liquid product is present in a range of about 5% to about 30% w/v.

Preferably said saliva activating agent in said liquid product is present in a range of about 5% to about 15% w/v.

Preferably said saliva activating agent in said liquid product is present at about 10% w/v.

Preferably said saliva activating agent in said liquid product is non-acidic.

Preferably said saliva activating agent in said liquid product is non-fermentable.

Preferably said saliva activating agent in said liquid product is a caries reducing agent.

Preferably said saliva activating agent in said liquid product is a plaque adhesion reducing agent.

Preferably said saliva activating agent in said liquid product is xylitol.

Preferably said oxidising agent in said liquid product is present in a range of about 200 PPM to about 1000 PPM in solution.

Preferably said oxidising agent in said liquid product is present in a range of about 300 PPM to about 700 PPM in solution.

Preferably said oxidising agent in said liquid product is present at about 500 PPM in solution.

Preferably said oxidising agent has a whitening and/or bleaching effect on teeth in said oral cavity.

Preferably said oxidising agent at least temporarily reduces or temporarily substantially alleviates anaerobic bacteria in said oral cavity.

Preferably said oxidising agent at least partially oxidises volatile sulphur compounds in said oral cavity, reducing instance of malodorous breath.

Preferably said oxidising agent is selected from any one or more of the following:
stabilised chlorine dioxide,
hydrogen peroxide,
zinc chloride.

Preferably said oxidising agent is stabilised chlorine dioxide.

Preferably said liquid product contains water in a range of about 80% to about 90% by weight.

Preferably said liquid product contains a non-fermentable flavouring agent.

Preferably said non-fermentable flavouring agent is menthol.

Preferably said liquid product also contains any one or more of the following:
a remineralising agent,
a stabilising agent,
a preservative.

Preferably said liquid product has a pH of about 7 to about 9.

Preferably said liquid product has a pH of about 7.5 to about 8.8

Preferably said liquid product has a pH of about 7.6 to about 8.5.

Preferably said liquid product is substantially non-coloured.

Preferably said liquid product is non-desiccating when introduced into said oral cavity.

Preferably said saliva activating agent in said powder product is present in a range of about 30% to about 80% w/w.

Preferably said saliva activating agent in said powder product is present in a range of about 40% to about 70% w/w.

Preferably said saliva activating agent in said powder product is present in a ranger of about 40% to about 60% w/w.

Preferably said saliva activating agent in said powder product is present at about 50% w/w.

Preferably said saliva activating agent in said powder product is non-acidic.

Preferably said saliva activating agent in said powder product is non-fermentable.

Preferably said saliva activating agent in said powder product is a caries reducing agent.

Preferably said saliva activating agent in said powder product is a plaque adhesion reducing agent.

Preferably said saliva activating agent in said powder product is xylitol.

Preferably said pH adjusting agent in said powder product is present in a range of about 1% to about 50% by weight.

Preferably said pH adjusting agent in said powder product is present in a range of about 20% to about 50% by weight.

Preferably said pH adjusting agent in said powder product is present is present in a range of about 30% to about 40% by weight.

Preferably said pH adjusting agent in said powder product is present at about 40% by weight.

Preferably said pH adjusting agent is sodium bicarbonate.

Preferably said remineralising agent in said powder product is present in a range of about 0.1% to about 30% by weight.

Preferably said remineralising agent in said powder product is present in a range of about 5% to about 20% by weight.

Preferably said remineralising agent in said powder product is present at about 10% by weight.

Preferably said remineralising agent in said powder product provides soluble mineral ions.

Preferably said remineralising agent is selected from any one or more of the following:
calcium glycerophosphate,
sodium fluoride.

Preferably said remineralising agent is calcium glycerophosphate.

Preferably said powder product has a pH of about 7 to about 9.

Preferably said powder product has a pH of about 7.8 to about 8.2.

Preferably said powder product has a pH of about 8.

Preferably said powder product substantially dissolves when introduced into said oral cavity.

Preferably said powder product is substantially non-abrasive when introduced into said oral cavity.

Preferably said powder product contains a non-fermentable flavouring agent.

Preferably said non-fermentable flavouring agent is menthol.

Preferably said powder product also contains any one or more of the following:
a stabilising agent,
a preservative,
a filler.

Preferably said saliva activating agent is the same in said liquid product and said powder product.

Preferably said saliva activating agent is xylitol in both said liquid product and said powder product.

Preferably said kit of parts also includes any one or more of the following:
a lozenge,
a flossing product
an oral spray.

Preferably said lozenge containing at least the following ingredients,
about 30% to about 95% by weight of a saliva activating agent, and a remineralising agent.

Preferably said lozenge product contains a pH adjusting agent.

Preferably said lozenge contains a non-fermentable flavouring agent.

Preferably said non-fermentable flavouring agent is selected from any one or more of the following:
liquorice extract,
menthol,
natural oil.

Preferably said lozenge contains at least one binding agent.

Preferably said binding agent is selected from any one or more of the following:
maltitol,
mannitol,
arabic gum.

Preferably said lozenge is selected from any one or more of the following,
a dissolvable lozenge,
a chewable tablet, whether gum based or otherwise.

In yet a further aspect the present invention consists in a method of improving the health of the oral cavity as herein described with particular reference to any one or more of the accompanying Examples and/or Figures.

In yet a further aspect the present invention consists in a product for improving the health of the oral cavity as herein described with particular reference to any one or more of the accompanying Examples and/or Figures.

In yet a further aspect the present invention consists in kit of parts as herein described with particular reference to any one or more of the accompanying Examples and/or Figures.

As used herein the term “and/or” means “and” or “or”, or both.

As used herein “(s)” following a noun means the plural and/or singular forms of the noun.

The term “comprising” as used in this specification means “consisting at least in part of”. When interpreting statements in this specification which include that term, the features, prefaced by that term in each statement, all need to be present, but other features can also be present. Related terms such as “comprise” and “comprised” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

The entire disclosures of all applications, patents and publications, cited above and below, if any, are hereby incorporated by reference.

To those skilled in the art to which the invention relates, many changes in construction and widely differing embodiments and application of the invention will suggest themselves without departing from the scope of the invention as defined in the appended claims. The disclosures and the descriptions herein are purely illustrative and are not intended to be in any sense limiting.

Other aspects of the invention may become apparent from the following description which is given by way of example only and with reference to the accompanying figures and examples.

### DETAILED DESCRIPTION OF THE INVENTION

The invention in general terms relates to compositions, methods and kits for the improvement of oral health.

In particular, in one aspect, the present invention relates to a method of improving the health of the oral cavity, comprising steps a) and b) in any order.

Step a) includes introducing into the oral cavity a liquid product containing at least about 5% to about 50% w/v of a saliva activating agent, an oxidising agent, and water. The liquid product is preferably held within the oral cavity for a period of time, prior to the liquid product being (at least in part) removed from the oral cavity, for example by spitting out the excess, but preferably not by washing or rinsing out the oral cavity, such that a residue of the liquid product remains in the oral cavity.

Step b) includes introducing into the oral cavity, preferably in conjunction with an oral tool (for example a dental brush/toothbrush, floss, gauze or finger dental brush), a product that is at least initially in powder form (“powder product”) containing at least about 30% to about 98% w/w of a saliva activating agent, a pH adjusting agent, and a remineralising agent. The powder product, (whether dissolved or otherwise) is then agitated in the oral cavity, preferably at least in part with the oral tool. The resultant product or products is then removed, at least in part, from the oral cavity. Removal for example is by spitting out the product, but, again, preferably not by washing or rinsing out the oral cavity. It is preferred that at least a residue of the powder product remains in the oral cavity.

Reference to “powder product” (in step b), should be understood to mean that the product may be a fine powder, granules or a mixture of particle sizes. In a more preferred form where the powder product dissolves when introduced into the oral cavity, the particle size can be selected to at least partially control the rate at which the product dissolves. It is particularly desirable for the powder product to dissolve in the oral cavity in order to reduce or prevent abrasion of the protective outer coating (enamel) of the teeth. In such a preferred form the powder product does not contain abrasive components, for example silica. While is some instances it may be beneficial to include abrasive components in the powder product, for example in a starter formulation to initially remove hardened material in the oral cavity, particularly the teeth, the most preferred formulation of the powder product dissolves in the oral cavity and it therefore non-abrasive (i.e. does not contain abrasive components). In addition, it preferable that the powder product dissolve in the oral cavity to allow maximal bioavailability of the minerals in the remineralising agent. Maximal bioavailability will allow of more effective remineralisation of the teeth.

The saliva activating agent in said liquid product should be in a range of about 5% to about 30% w/v, more preferably in a range of about 5% to about 15% w/v, even more preferably it should be present at about 10% w/v.

The saliva activating agent in the powder product in a range of about 30% to about 80% w/w, preferably about 40% to about 70% w/w, preferably about 40% to about 60% w/w, most preferably about 50% w/w.

In preferred forms the saliva activating agent is non-acidic and/or is a caries reducing agent and/or is a plaque adhesion reducing agent and/or is non-fermentable. In the most preferred form the saliva activating agent is xylitol in the liquid product and/or the powder product.

Saliva acts as a natural defence in the oral cavity, by breaking down food, buffering acids from diet and bacteria to maintain a healthy neutral or near neutral pH in the oral cavity (i.e. about pH 6.8-7.2), providing bioavailable minerals (dissolved in the saliva) to remineralise teeth and providing oxygen and antibodies that help fight pathogenic bacteria in the oral cavity and around the gum-line. It is also a lubricant in the oral cavity. While saliva is naturally occurring and production is usually stimulated in response to eating (stimulated saliva), environmental issues, for example alcohol, caffeine, smoking and diet, and natural or induced physiological variation, affect the levels of saliva produced and the quality of that saliva (i.e. the ability to perform the beneficial functions discussed above). It has therefore been found to be beneficial to activate/stimulate the production of saliva in the oral cavity and in addition enhance/encourage the saliva's ability to provide the beneficial functions discussed above. While not wishing to be bound by theory, it is believed the methods and products of the invention act by increasing the production of saliva in the oral cavity. Further the saliva either produced as a result of the method/product or occurring naturally is enhanced by increasing the buffering capacity of the saliva, and/or increasing the mineral content in the saliva and/or increasing the plaque-adhesion reducing ability of the saliva and/or increasing the oxygen content in the saliva. Such saliva is referred to herein as “enhanced saliva”. By contrast, some oral health products are desiccating (i.e. they lower the levels of saliva in the oral cavity), and as a result the beneficial effects of encouraging and activating saliva (and/or enhanced saliva) do not occur in these products.

Stimulated saliva (produced naturally when eating) is alkaline (ph 7.8-8.5) and contains bicarbonate, calcium and phosphate ions to buffer acids from the diet and bacteria that break down the food. These ions also help remineralise teeth after the acid attack. Ideally the oral environment is maintained in a neutral or near neutral pH (6.8-7.2) most of the time. Low pH (below about 5.5) in the oral cavity will cause demineralisation of the teeth, as it is believed the minerals will be leached out of the teeth at such low pH. In addition, at acidic pH (lower than neutral) remineralisation will not occur (or be limited to a low level). With excessive dietary intake of acids, partially due to changing dietary habits, and the increased use of saliva inhibiting agents (alcohol, caffeine, drugs, smoking, desiccating oral agents) many people do not get adequate protection against the attack of bacterial biofilm and dietary acids from their own naturally produced/stimulated saliva. The products and/or methods of the invention mimic and enhance the functions and ingredients of stimulated saliva. The products and/or methods of the invention have been found to be surprisingly effective in the reduction of detrimental bacteria in the oral cavity which lead to caries and/or malodorous breath.

The oxidising agent in the liquid product has a mild whitening and/or bleaching effect on teeth in the oral cavity. The oxidising agent at least temporarily reduces or temporarily substantially alleviates the effect of anaerobic and pathogenic bacteria in the oral cavity. A further beneficial effect of the oxidising agent is to at least temporarily reduce or temporarily alleviate malodours in the oral cavity. While not wishing to be bound by theory, it is believed at least one of the beneficial effects of the oxidising agent is to oxidise volatile sulphur compounds (VSCs) in the mouth which cause malodours, When oxidised, it is believed the malodours of VSCs are eliminated or at least ameliorated. Mechanisms by which VSCs occur in the oral cavity include introduction in odorous food, for example garlic, production by bacteria in the oral cavity, bacteria in plaque and below the gum-line and rotting food.

By temporary, it should be understood the effect on the anaerobic bacteria and malodours should last at least 10 minutes, more preferably at least an hour. The effect is likely to last for a maximum of 12 hours. However, it would be apparent to a person skilled in the art that the existing oral health, eating habits, smoking, and individual physiological characteristics of the subject will affect the amount and the time for which the effects of the anaerobic bacteria and malodours are alleviated/reduced. However, there should at least be some beneficial effect in substantially all subjects.

Examples of suitable oxidising agents can be selected from anyone or more of stabilised chlorine dioxide and/or hydrogen peroxide and/or zinc chloride. However the preferred oxidising agent is stabilised chlorine dioxide. Stabilised chlorine dioxide has the additional benefits of being anti-bacterial, anti-viral and anti-fungal. In addition, stabilised chlorine dioxide is believed to be a superior oxidising agent, for example in comparison to chorine. Stabilised chlorine dioxide has also believed to be safer for human use than other common oxidising agents, for example chlorine. Stabilised chlorine dioxide is in addition, non-desiccating in the oral cavity. Stabilised chlorine dioxide is alternatively known as sodium chlorite. Stabilised chlorine dioxide/sodium chlorite may additionally contain bicarbonate in less than 1% w/w to stabilise it. The oxidising agent should be present in the liquid product in a range of about 200 PPM to about 1000 PPM of the liquid product, preferably about 300 PPM to about 700 PPM, most preferably about 500 PPM of the liquid product. However, it will be apparent the range will be at least partially dependent on the oxidising agent selected.

In addition, the liquid product is preferably substantially non-coloured in order to reduce and/or prevent staining of the oral cavity (in particular the teeth). It particular, the liquid product is preferably substantially non-coloured in order to maximise (or at least not work against) the mild whitening and/or bleaching effect of the oxidising agent.

The liquid product will also contain water in a range of 80% to 90% by weight. It is preferred the water is deionised and/or otherwise purified.

The remineralising agent should be present in the powder product in a range of about 0.1% to about 30% by weight, preferably about 5% to about 20% by weight, more preferably about 10% by weight. The remineralising agent is selected to provide mineral ions which are available for absorption in the oral cavity, preferably by the teeth. The remineralising agent should be selected to provide calcium, fluoride and/or phosphate ions. The pH of the oral cavity (which is affected/regulated by the liquid and powder products) will also affect the availability of the mineral ions. The pH of the liquid and powder products is therefore important to the effectiveness of the remineralising agent. As discussed above, low pH (below about 5.5) in the oral cavity will cause demineralisation of the teeth, as it is believed the minerals will be leached out of the teeth at such low pH. In addition, at acidic pH (lower than neutral) remineralisation will not occur (or be limited to a low level). Suitable remineralising agents include sodium fluoride and calcium glycerophosphate. However, in a preferred embodiment the remineralising agent is calcium glycerophosphate. Calcium glycerophosphate is particularly preferred because it is considered a safe food grade ingredient. In addition calcium glycerophosphate dissolves quickly to provide bioavailable calcium and phosphate ions which are the minerals naturally present in stimulated saliva to remineralise teeth. In addition the ions may aid in buffering the pH of the oral cavity.

The liquid product should be neutral or basic, preferably having a pH of about 7 to about 9, preferably about 7.5 to about 8.8, more preferably about 7.6 to 8.5. The pH of the powder product should preferably be about 7 to about 9, preferably about 7.8 to about 8.2, more preferable about 8. As noted above the pH of oral cavity will affect the efficacy of the remineralising agent in the powder product. In a preferred aspect, where the liquid product is used prior to the powder product, the liquid product acts in conjunction with the powder product by adjusting/buffering the pH in the oral cavity to neutral or near neutral (about pH 6.8-7.2) prior to introduction of the powder product, including the remineralising agent.

The pH adjusting agent in said powder product in a range of about 1% to about 50% by weight, preferably about 20% to about 50% by weight, preferably about 30% to about 40% by weight, more preferably about 40% by weight. The amount of the pH adjusting agent can be selected to provide the required pH when the product is introduced into the oral cavity and to providing a buffering action to maintain (at least temporarily) the oral cavity at the required pH. The pH adjusting agent in the powder product also adjusts the pH in the oral cavity and buffers the pH to maximise the effect of the remineralising agent. This is particularly important where the liquid product is not used, or where the oral cavity is very acidic. In addition, an acidic environment in the oral cavity is known to promote the growth of harmful/detrimental bacteria in the biofilm.

Any suitable pH adjusting agents may be used in the invention. However, in a preferred embodiment the pH adjusting agent is sodium bicarbonate. Sodium Bicarbonate is particularly preferred as a pH adjusting agent/buffering agent because it is a food grade substance, which is considered safe for human consumption (at the levels used in the invention). It is also naturally occurring in stimulated saliva.

Flavouring agents can also be added to the products to make the method more pleasant for the subject. Such flavouring agent should be non-fermentable in order to maintain the beneficial effects of the liquid product. For example, flavouring such as sugars, which are consumed by oral bacteria and/or are likely to have a detrimental effect on the oral cavity are preferably not used. The preferred flavouring agents include menthol, liquorice extracts, cinnamon oil, grapefruit oil, and other natural oils. The most preferred favouring agent is menthol.

Reference to “non-fermentable” should be taken to include low levels of fermentation and should be taken to mean a substance that is not broken down (or only broken down to a small extent) by bacteria to produce an acidic by-product. Glucose and fructose are examples of highly fermentable carbohydrates. Xylitol is an example of a non-fermentable carbohydrate.

The products may optionally also contain further components, for example the liquid product may additionally contain a remineralising agent, and both liquid and powder products may contain any one or more of: a stabilising agent, a preservative, a filler.

The liquid product and powder product are preferably non-desiccating when introduced into said oral cavity. As previously noted, the saliva activating agent stimulates and/or enhances saliva production. It is therefore undesirable for other components of the products to have a desiccating effect, such that the overall effect of the products would be desiccating. Some components may have a minor desiccating effect but they should be selected to minimise this. Desiccating agents, for example alcohol and detergents, have previously often been added to oral/dental hygiene products as cleaning agents and/or to keep manufacturing costs at minimum and/or prolong their shelf life of products and/or dissolve other ingredients and/or create a physical reaction in the mouth that may be used to market the product (e.g. the stinging/tingling effect of alcohols in the oral cavity, or the bubbling and foaming of detergents). The inventors have surprisingly found, such desiccating agents, rather than having the previously believed beneficial properties on oral heath actually have a detrimental effect on the health of the oral cavity (including teeth). In particular, many desiccating ingredients were previously thought to clean the oral cavity. However, the inventors have surprisingly found such harsh cleaning agents in fact can pose a detrimental effect, rather than a beneficial effect, on the health of the oral cavity.

The inventors have found ingredients and components in previous oral/teeth care products which are acidic, desiccating and cause erosion act against the natural defences of saliva, on the teeth and mouth. Such ingredients and components create an oral environment that is less favourable for the growth of protective biofilm and more favourable for the growth of harmful biofilm containing harmful bacteria. A dry, acidic, low oxygen environment will promote the growth of harmful biofilm containing bacteria that thrive in such an environment. These bacteria increase the risk of an individual to dental caries and periodontal disease, both of which are associated with or caused by detrimental bacterial biofilm.

The components of the liquid product and powder product act together in a synergistic way to promote a beneficial biofilm in the oral cavity. The components act in the ways described about to create a combined effect which decreases the number of detrimental bacteria (including anaerobic bacteria) in the biofilm and promotes beneficial bacteria in their place. It is believed previous oral health products attacked all bacteria (both detrimental and beneficial) in the oral cavity rather than encouraging the formation of a protective biofilm of beneficial bacteria.

The period of time the liquid product is preferable retained in the oral cavity is at least 10 seconds, preferably between 10 seconds to 2 minutes, preferable for 1 minute. The time is selected to allow the active ingredients sufficient contact time to have a beneficial effect, for example to neutralise acids in the oral cavity and at least partially oxidise volatile sulphur compounds and attack anaerobic bacteria, fungus and viruses that may be present in said oral cavity and around the gum-line.

The liquid product and/or the powder product should preferably be removed from the oral cavity by expectorating. The oral cavity is preferably not rinsed (for example with water) following the removal of the liquid product and/or the powder product. Any residue of the products should preferably remain in the oral cavity to continue to provide the beneficial properties, rather than being removed by rinsing.

The powder product, whether dissolved or otherwise, is preferably agitated/spread around the oral cavity using an oral tool, for example a dental brush/toothbrush, floss, gauze and/or finger dental brush (although a finger may be sufficient). The agitation should spread the product over any one or more of the teeth, gums, tongue and cheek(s), preferable at least the teeth but more preferably substantially the entire oral cavity.

In the most preferred aspect of the method step a) is followed by step b). In this order the liquid product can be used to remove debris from the oral cavity, for example particles of food, to prepare for the powder product. In addition, the liquid product in step a) may neutralise acid in said oral cavity prior to carrying out step b). It is highly beneficial to neutralise acid in the oral cavity prior to application of the powder product, so that the saliva activating agent and remineralising agent in the powder product can have optimal action/benefits. It is also desirable to apply the liquid product prior to the powder product so that the residue of the remineralising agent will remain in the oral cavity after treatment to continue acting on the teeth. If the liquid product is used after the powder product the residue of the remineralising agent will be rinsed away.

The method (including steps a) and b)) is preferably carried out on at least a daily basis. However, more preferably steps a) and b) are repeated at least twice in a 24 hour period, for example, in the morning and in the evening. However, the personal choice of the subject and/or the existing oral health of the subject may lead to the method being carried out on a less frequent or more frequent basis.

In addition to steps a) and b) the method optionally includes introducing into said oral cavity a lozenge product containing at least the ingredients: 30% to 95% by weight of a saliva activating agent, (for example, but not limited to, xylitol) and a remineralising agent (for example, but not limited to, calcium glycerophosphate and/or sodium fluoride). The lozenge will additionally preferably contain a pH adjusting agent (for example, but not limited to, sodium bicarbonate). The lozenge will also preferably contains a non-fermentable flavouring agent (for example, but not limited to liquorice extract, menthol, natural oil(s)) to encourage use and make a more pleasant treatment experience. The lozenge product may also contain at least one binding agent, depending on the consistency/texture of product required. For example maltitol and/or mannitol may be used to create a hard candy and arabic gum to create a softer or chewable product.

Reference to lozenge product should be taken to mean a dissolvable lozenge, and/or a chewable tablet, for example gum based, or otherwise. Chewable tablets are particularly preferred, as the chewing action stimulates saliva to be produced in the oral cavity, which further complements the action of the liquid product and powder products which also stimulate production of saliva.

Where the method includes a lozenge product it is preferably introduced into the oral cavity at least once in a 24 hour period, preferable at least three times in a 24 hour period. For example, the lozenge product may conveniently be used after eating, drinking or smoking. Where the lozenge product is used after eating, drinking or smoking it is preferably introduced into said oral cavity within about 30 minutes of eating or drinking, more preferably within about 10 minutes of eating or drinking. It is preferable to use the lozenge product as soon after eating and/or drinking and/or smoking as is practical as it is important to maintain a neutral to slightly alkaline pH in the oral cavity. As a general rule, bacteria that cause most oral disease thrive in an acidic environment, whereas beneficial or benign bacteria thrive in a neutral pH (or near neutral pH).

Another aspect of the invention should be considered to be a product for improving the health of the oral cavity, comprising about 30% to about 98% w/w of a saliva activating agent, a pH adjusting agent, an oxidising agent and a remineralising agent wherein examples of saliva activating agents, pH adjusting agents, oxidising agent and remineralising agents are discussed above. The saliva activating agent activates stimulated saliva in said oral cavity. The pH adjusting agent rapidly adjusts the pH of said saliva to said pH range of about 7.8 to 8.2 and adjusts and buffers the oral cavity to pH range of about 6.8-7.2. The remineralising agent provides an excess of at least one mineral to at least reduce demineralisation of at least one tooth in said oral cavity. The product may take the form of any one or more of: a powder product, a mouth wash product (liquid product) a lozenge or tablet (whether gum-based or otherwise), and/or a floss.

In a further aspect the present invention consists in a product for improving the health of the oral cavity, comprising about 30% to about 98% w/w of a saliva activating agent, a pH adjusting agent, and a remineralising agent. The product has a pH of about 7.8 to 8.2. The product is initially in solid form for ease of application to a tool (for example a dental brush/toothbrush, floss, gauze and/or finger dental brush) and application of the product applied to the tool to the oral cavity. However, the product dissolves when introduced into said oral cavity, to minimise non-desirable abrasive action, which can where away tooth enamel.

In yet another aspect the present invention consists in a kit of parts for improving the health of the oral cavity, comprising a powder product and a liquid product, as previously described. The kit of parts may additionally optionally include a lozenge or tablet (whether gum-based or otherwise) and/or a floss and/or an oral spray. The oral spray preferably contains any one or more of the following components: a saliva activating agent (preferably about 10 to about 70% w/v), an oxidising agent (preferably about 100 PPM to about 1000 PPM), a pH adjusting agent (preferably about 1 to about 50% w/v), a remineralising agent (preferably about 1 to about 20% w/v). Preferably the oral spray contains a saliva activating agent and an oxidising agent. Suitable saliva activating agents, oxidising agents and pH adjusting agents have previously been discussed in relation to the powder and liquid products.

### EXAMPLES

### Example 1

Example formulations of Liquid Product are shown in Table 1, below.

TABLE 1

| Formulation 1 | Formulation 2 |
|---|---|
| Purified Water | Purified Water |
| Xylitol 10% w/v | Xylitol 10% w/v |
| Stabilized chlorine dioxide | Stabilized chlorine dioxide |
| (500 PPM in solution) | (1000 PPM in solution) |
| Menthol 6g/l | Menthol 6 g/l w/v |
| | Calcium Glycerophosphate 5% w/v |

Steps for manufacture:
a. 500 PPM (parts per million) is the desired concentration of the Stabilized Chlorine Dioxide (SCD) in the solution. 10% (weight to volume) milled xylitol (1 g/10 ml of solution) is added and mixed with 6 g/l of milled Menthol crystal.
b. Shake and mix the solution very well, then sieve any un-dissolved crystals leaving a near clear
c. The pH is 7.6-8.5
   Calcium Glycerophosphate may also be added to the liquid product.

### Example 2

Example formulations of Powder Product are shown in Table 2, below.

TABLE 2

Formulation 1
Xylitol powder 50% w/w
Sodium Bicarbonate 40% w/w
Calcium Glycerophosphate 10% w/w
Menthol 0.035% w/w

50% Xylitol powder (finely milled from Xylitol crystals), 40% Sodium Bicarbonate, 10% Calcium Glycerophosphate and 0.035% Crystal Menthol powder (finely milled) are milled, sieved and mixed together to create a uniform white powder. The pH of the powder is 8 (7.8-8.2).

### Example 4

Example formulations of optional products in method are shown in Table 3.

TABLE 3

| Example formulation of | | |
|---|---|---|
| chewable | Example formulation | Example formulation |
| tablet/lozenge | of dissolvable lozenge | of oral spray |
| Xylitol 0.6-0.8 g | Xylitol 0.6-0.8 g | Purified Water |
| Calcium | Calcium | Vegetable Glycerine |
| glycerophosphate 0.2 g | glycerophosphate 0.2 g | |
| Liquorice extracts | Liquorice extracts | Methylcellulose |
| Arabic gum | Maltitol/mannitol | Stabilised Chlorine |
| | | Dioxide 0.5-1% w/v |
| Natural oil(s) for flavours | Natural oil(s) | Xylitol 20-50% w/v |
| | for flavours | |
| | | Sodium Bicarbonate |
| | | 10-20% w/v |
| | | Natural flavours |
| | | selected from: |
| | | menthol oil, |
| | | cinnamon oil, |
| | | grapefruit oil |

### Example 3

### Challenge Testing of Powder Product Against Organism Steptococcus mutans

A challenge test was conducted under laboratory conditions for the dental powder product (formulation 1). The test was a bacterial challenge test against the organism Streptococcus mutans and was further analysed over a longer period of time. (Streptococcus mutans is commonly found in the mouth, and is regarded as a significant contributor to tooth decay.)

### Methodology for Challenge Test:

The challenge method used was based on B.S.3286: 1960 “Method for Laboratory Evaluation of Disinfectant Activity of Quaternary Ammonium Compounds by Suspension Test Procedure”. This standard describes a test procedure for the laboratory evaluation of the activity of disinfectants, in which a suspension of viable microorganisms is added to solutions containing a pre-determined concentration of the disinfectant and the numbers of organisms surviving after a period of time is determined. A 1:5 solution of dental powder product with sterile deionised water was used for the challenge testing.

A neutralising inactivation test had been previously performed to ascertain the ability of the neutralising buffer to stop the action of any antimicrobial agents present in the sample. The results of the analysis showed the product solution was neutralised.

The organism Streptococcus mutans (ATCC 25175) was used for the challenge testing. The culture was grown on Tryptic Soy Agar and a suspension made to achieve an approximate level of 5.0×106 colony forming units per ml (cfu/ml) when inoculated into the solution under test. (The identity of the bacterium was revalidated using an API Strep test.)

0.1 ml of this culture was inoculated into 10 ml volumes of a blank solution (neutralising buffer) and the product test solution and mixed.

A modification will be made to the method by the addition of 10 ml of the 1:5 solution of dental powder product solution (“1:5 powder solution”) each day, for 4 days. (The blank solution would have 10 ml of Neutralising buffer added.)

The solutions were then sampled at:
T=0 (Day 0)
T=1 (At 1 day, prior to adding 10 ml of the 1:5 powder solution)
T=2 (2 hours after adding 10 ml of the 1:5 powder solution on Day 1)
T=3 (At 2 days, prior to adding 10 ml of the 1:5 powder solution)
T=4 (2 hours after adding 10 ml of the 1:5 powder solution on Day 2)
T=5 (At 3 days, prior to adding 10 ml of the 1:5 powder solution)
T=6 (2 hours after adding 10 ml of the 1:5 powder solution on Day 3)
T=7 (At 4 days, prior to adding 10 ml of the 1:5 powder solution)
T=8 (2 hours after adding 10 ml of the 1:5 powder solution on Day 4)

The results of the test challenge are shown in Table 4.

TABLE 4

| | Streptococcus mutans ATCC 25175 Population Levels | | | | | | | | |
|---|---|---|---|---|---|---|---|---|---|
| | | Day 1 | | Day 2 | | Day 3 | | Day 4 | |
| | | T = 1 | T = 2 | T = 3 | T = 4 | T = 5 | T = 6 | T = 7 | T = 8 |
| | | cfu/ml | cfu/ml | cfu/ml | cfu/ml | cfu/ml | cfu/ml | cfu/ml | cfu/ml |
| | | Prior to | 2 hrs after | Prior to | 2 hrs after | Prior to | 2 hrs after | Prior to | 2 hrs after |
| | Day 0 | 10 mls of | 10 mls of | 10 mls of | 10 mls of | 10 mls of | 10 mls of | 10 mls of | 10 mls of |
| | T = 0 | fresh sol'n | fresh sol'n | fresh sol'n | fresh sol'n | fresh sol'n | fresh sol'n | fresh sol'n | fresh sol'n |
| Solution | cfu/ml | added | added | added | added | added | added | added | added |
| Neutralising | 2.5 × 10⁶ | 3.2 × 10⁶ | 1.4 × 10⁶ | 1.6 × 10⁶ | 6.5 × 10⁵ | 5.5 × 10⁵ | 2.7 × 10⁵ | 2.8 × 10⁵ | 2.1 × 10⁵ |
| Buffer Solution | | | | | | | | | |
| 1:5 powder | 2.7 × 10⁶ | 2.0 × 10⁴ | 1.2 × 10⁴ | 2.8 × 10² | 1.7 × 10² | 70 | 13 | 20 | 20 |
| product solution | | | | | | | | | |

Limit of detection: 10 colony -forming units (cfu) per millilitre (ml)

### Challenge Test Comments:

The organism populations would have naturally become diluted by approximately half each time fresh solution was added. Even when this is taken in to account, every 24 hours there appeared to be a much higher overall population drop in the powder product solution than in the neutralising buffer solution.
Simple dilution over the 5-day period accounted for a 1-log reduction in the Streptococcus mutans population over the 5 days in the Neutralising Buffer solution, but a 5-log reduction was observed in the Streptococcus mutans population present in the powder product solution.
These results strongly suggest that the powder product has had a significant effect on reducing the population of Streptococcus mutans over a sustained period of time in a controlled laboratory test regime. This testing protocol does not take in to account what effect residual food, saliva or other competing microorganisms would have on the effectiveness of the product in a ‘real life’ setting.

### Example 4

### Challenge Testing of Powder Product and Liquid Product Against Organisms Steptococcus mutans and Porphyromonas gingivalis

The powder product and the liquid product were challenge tested against the organisms Streptococcus mutans and Porphyromonas gingivalis. (Both of these bacteria are commonly found in the mouth, and are regarded as significant contributors to tooth decay and bad breath.) Xylitol and Bicarbonate of Soda (Sodium Bicarbonate) were also tested in the same way for comparison. The samples tested are shown in Table 5.

TABLE 5

| Sample Type | Solution Test Strength |
|---|---|
| Powder product (formulation 1) | 1:5 |
| Liquid product (formulation 1) | Neat |
| Xylitol powder | 1:5 |
| Bicarbonate of Soda powder | 1:5 |

### Methodology for Challenge Test:

The challenge method used was based on B.S.3286: 1960 “Method for Laboratory Evaluation of Disinfectant Activity of Quaternary Ammonium Compounds by Suspension Test Procedure”. This standard describes a test procedure for the laboratory evaluation of the activity of disinfectants, in which a suspension of viable microorganisms is added to solutions containing a pre-determined concentration of the disinfectant and the numbers of organisms surviving after a period of time is determined. A 1:5 solution of the powders with sterile deionised water was used for the challenge testing. The liquid product was used neat. The following organisms were used for the challenge testing:

| | |
|---|---|
| Streptococcus mutans | ATCC 25175 |
| Porphyromonas gingivalis | ATCC 3327 |

The cultures were grown on Tryptic Soy Agar and a suspension made to achieve an approximate level of 2.0×10⁶-2.0×10⁷ colony-forming units per ml (cfu/ml) when inoculated into the solutions under test. 0.1 ml of these cultures were inoculated into 10 ml volumes of a blank solution (neutralising buffer) and the sample solutions and mixed.

A modification will be made to the method by the addition of 10 ml of each solution each day, for 4 days. (The blank solution would have 10 ml of Neutralising buffer added.)

The solutions were then sampled at:
T=0 (Day 0)
T=1 (At 1 day, prior to adding 10 ml of fresh solution)
T=2 (2 hours after adding 10 ml of fresh solution on Day 1)
T=3 (At 2 days, prior to adding 10 ml of fresh solution)
T=4 (2 hours after adding 10 ml of fresh solution on Day 2)
T=5 (At 3 days, prior to adding 10 ml of fresh solution)
T=6 (2 hours after adding 10 ml of fresh solution on Day 3)
T=7 (At 4 days, prior to adding 10 ml of fresh solution)
T=8 (2 hours after adding 10 ml of fresh solution on Day 4)

The results of the test challenge against Streptococcus mutans are shown in Table 6.

TABLE 6

| | Streptococcus mutans ATCC 25175 Population Levels | | | | | | | | |
|---|---|---|---|---|---|---|---|---|---|
| | | Day 1 | | Day 2 | | Day 3 | | Day 4 | |
| | | T = 1 | T = 2 | T = 3 | T = 4 | T = 5 | T = 6 | T = 7 | T = 8 |
| | | cfu/ml | cfu/ml | cfu/ml | cfu/ml | cfu/ml | cfu/ml | cfu/ml | cfu/ml |
| | | Prior to | 2 hrs after | Prior to | 2 hrs after | Prior to | 2 hrs after | Prior to | 2 hrs after |
| | Day 0 | 10 mls of | 10 mls of | 10 mls of | 10 mls of | 10 mls of | 10 mls of | 10 mls of | 10 mls of |
| | T = 0 | fresh sol'n | fresh sol'n | fresh sol'n | fresh sol'n | fresh sol'n | fresh sol'n | fresh sol'n | fresh sol'n |
| Solution | cfu/ml | added | added | added | added | added | added | added | added |
| Neutralizing | 2.0 × 10⁷ | 1.9 × 10⁷ | 8.1 × 10⁶ | 7.3 × 10⁶ | 4.6 × 10⁶ | 5.0 × 10⁶ | 4.3 × 10⁶ | 2.8 × 10⁶ | 3.2 × 10⁶ |
| Buffer Solution | | | | | | | | | |
| Powder product | 1.3 × 10⁷ | 2.2 × 10⁴ | 2.2 × 10⁴ | 1.8 × 10² | 1.6 × 10² | 2.0 × 10² | 10 | 30 | <10 |
| solution | | | | | | | | | |
| Liquid product | 2.1 × 10⁷ | 7.3 × 10⁶ | 2.4 × 10⁶ | 10 | <10 | <10 | <10 | <10 | <10 |
| solution | | | | | | | | | |
| Xylitol solution | 1.5 × 10⁷ | 7.8 × 10⁶ | 5.2 × 10⁵ | 1.0 × 10⁵ | 2.6 × 10⁴ | 1.0 × 10⁴ | 1.6 × 10⁴ | 30 | <10 |
| Bicarbonate of | 1.5 × 10⁷ | 1.8 × 10⁶ | 3.4 × 10⁵ | 6.7 × 10⁴ | 3.2 × 10⁴ | 7.7 × 10³ | 2.8 × 10³ | 1.2 × 10² | 10 |
| Soda solution | | | | | | | | | |

Limit of detection: 10 colony -forming units (cfu) per millilitre (ml)

Challenge Test Comments for Streptococcus mutans:
The organism populations would have naturally become diluted each time fresh solutions were added. Even when this is taken in to account, every 24 hours there appeared to be a much higher overall population drop in the powder product solution and the liquid product, especially the liquid product, than in the neutralising buffer solution and the xylitol and bicarbonate of soda solutions.
Simple dilution over the 5-day period accounted for a 1-log reduction in the Streptococcus mutans population over the 5 days in the Neutralising Buffer solution, but a greater than 7-log reduction was observed in the Streptococcus mutans population present in the powder product solution and the liquid product.
The Xylitol and bicarbonate of soda also had a greater than 7-log reduction and 6-log reduction respectively over the 5 days, but the populations persisted in higher levels for longer during this period. These results strongly suggest that the powder product and the liquid product have had a significant effect on reducing the populations of Streptococcus mutans over a sustained period of time in a controlled laboratory test regime. This testing protocol does not take in to account what effect residual food, saliva or other competing microorganisms would have on the effectiveness of the product in a ‘real life’ setting.

The results of the test challenge against Porphyromonas gingivalis are shown in Tables 7 and 8.

TABLE 7

| | Porphyromonas gingivalis ATCC 3327 Population Levels | | | | | | | | |
|---|---|---|---|---|---|---|---|---|---|
| | | Day 1 | | Day 2 | | Day 3 | | Day 4 | |
| | | T = 1 | T = 2 | T = 3 | T = 4 | T = 5 | T = 6 | T = 7 | T = 8 |
| | | cfu/ml | cfu/ml | cfu/ml | cfu/ml | cfu/ml | cfu/ml | cfu/ml | cfu/ml |
| | | Prior to | 2 hrs after | Prior to | 2 hrs after | Prior to | 2 hrs after | Prior to | 2 hrs after |
| | Day 0 | 10 mls of | 10 mls of | 10 mls of | 10 mls of | 10 mls of | 10 mls of | 10 mls of | 10 mls of |
| | T = 0 | fresh sol'n | fresh sol'n | fresh sol'n | fresh sol'n | fresh sol'n | fresh sol'n | fresh sol'n | fresh sol'n |
| Solution | cfu/ml | added | added | added | added | added | added | added | added |
| Neutralising | 2.4 × 10⁶ | 2.0 × 10⁶ | 8.1 × 10⁵ | 5.0 × 10⁵ | 2.8 × 10⁵ | 2.0 × 10⁵ | 1.4 × 10⁵ | 1.2 × 10⁵ | 8.7 × 10⁴ |
| Buffer Solution | | | | | | | | | |
| Liquid product | 2.4 × 10⁶ | <10 | <10 | <10 | <10 | <10 | <10 | <10 | <10 |
| solution | | | | | | | | | |
| Xylitol solution | 2.6 × 10⁶ | 9.1 × 10³ | 1.9 × 10³ | 20 | <10 | <10 | <10 | <10 | <10 |
| Bicarbonate of | 2.5 × 10⁶ | 5.9 × 10⁵ | 3.1 × 10⁵ | 1.2 × 10⁵ | 1.1 × 10⁵ | 2.4 × 10⁴ | 3.3 × 10⁴ | 4.8 × 10³ | 2.8 × 10³ |
| Soda solution | | | | | | | | | |

Limit of detection: 10 colony -forming units (cfu) per millilitre (ml)

TABLE 8

| | Porphyromonas gingivalis ATCC 3327 Population Levels | | | | | | | | |
|---|---|---|---|---|---|---|---|---|---|
| | | Day 1 | | Day 2 | | Day 3 | | Day 4 | |
| | | T = 1 | T = 2 | T = 3 | T = 4 | T = 5 | T = 6 | T = 7 | T = 8 |
| | | cfu/ml | cfu/ml | cfu/ml | cfu/ml | cfu/ml | cfu/ml | cfu/ml | cfu/ml |
| | | Prior to | 2 hrs after | Prior to | 2 hrs after | Prior to | 2 hrs after | Prior to | 2 hrs after |
| | Day 0 | 10 mls of | 10 mls of | 10 mls of | 10 mls of | 10 mls of | 10 mls of | 10 mls of | 10 mls of |
| | T = 0 | fresh sol'n | fresh sol'n | fresh sol'n | fresh sol'n | fresh sol'n | fresh sol'n | fresh sol'n | fresh sol'n |
| Solution | cfu/ml | added | added | added | added | added | added | added | added |
| Neutralising | 7.3 × 10⁶ | 5.7 × 10⁶ | 3.5 × 10⁶ | 1.0 × 10⁶ | 5.5 × 10⁵ | 7.4 × 10⁴ | 5.3 × 10⁴ | 1.4 × 10³ | 1.9 × 10³ |
| Buffer Solution | | | | | | | | | |
| Powder product | 6.7 × 10⁶ | 6.8 × 10⁵ | 1.5 × 10⁵ | 4.3 × 10² | 1.8 × 10² | 50 | 20 | <10 | <10 |
| solution | | | | | | | | | |

Challenge Test Comments for Porphyromonas gingivalis:
The organism populations would have naturally become diluted each time fresh solutions were added. The opening and shaking of the solutions each time would′ve also introduced oxygen to an extent. (This likely explains the difference in level of ‘die-off’ between the two bacterial types.)
Simple dilution over the 5-day period accounted for a 1.5 to 3.5-log reduction in the Porphyromonas gingivalis population over the 5 days in the Neutralising Buffer solution, but a greater than 6-log reduction was observed in the Porphyromonas gingivalis population present in the powder product solution and the liquid product.
The Xylitol also had a greater than 6-log reduction over the 5 days, appearing to be reasonably effective against the anaerobe.
The bicarbonate of soda did not appear to be effective on its own against the Porphyromonas gingivalis population, having a similar level of ‘die off’ to the neutralising buffer control.
These results strongly suggest that the powder product solution and the liquid product have had a significant effect on reducing and eliminating the populations of Porphyromonas gingivalis over a sustained period of time in a controlled laboratory test regime. This testing protocol does not take in to account what effect residual food, saliva or other competing microorganisms would have on the effectiveness of the product in a ‘real life’ setting.

### Example 5

Trials were conducted to assess the effect of the combination of the liquid product and the powder product on the bacteria in the oral biofilm of patients in a “real life” setting.

20 patients of different age groups were included in the trial. At the start of the trial ATP swabs were used to test for the presence of bacteria in the oral cavity. The level of ATP on each swab was measured digitally and displayed as a number. In addition the oral pH was measured. Each participate in the trial was then instructed on how to use the method and products of the invention. They were told to hold 5 ml of the liquid product in the oral cavity for 1 minute, while moving the liquid product around the oral cavity, then expectorate the liquid product, without rinsing (for example with water).

Following use of the liquid product, the participants were instructed to dip a soft slightly moist toothbrush in the powder product, and brush around the teeth and gums for 2 minutes. The participants were instructed to expectorate excess saliva during and after brushing, but not to rinse out the mouth (for example with water).

The participants were instructed to use the liquid product followed by the powder product twice daily. They were also instructed to stop using any other dental products for the period of the trial. They were, however, allowed to continue their normal daily habits to ensure that the real effect of the products and methods of the invention could be ascertained. Thus, an individual's smoking/drinking habits and diet were unchanged.

At the end of the trial ATP swabs were used to test for the presence of bacteria in the oral cavity. The level of ATP on each swab was measured digitally and displayed as a number. The average reduction in the ATP measured the oral cavity was 84% within 2-3 months for most people. Higher levels of ATP are an indication of the presence of a high level of detrimental bacteria in the biofilm in the oral cavity. The more active the bacteria in the swab (i.e. the higher the metabolism of sugar), the higher the reading of ATP on the swab.

One subject had 95% reduction in the bacteria tested and one subject had 25% reduction in the bacteria tested. This variance can be attributed to a number of factors, for example the subject's eating habits, smoking, alcohol consumption, physical variation between subjects (for example natural pH of mouth) and compliance with the instructed method of application of the products. Nevertheless, taking all these variances into account all the subjects in the trial had a reduction in the detrimental bacteria in their oral biofilm, with an average of 84% reduction in the bacteria tested.

The results show the surprising efficacy of the combination of the liquid product and the powder product. The results show good improvement in the level of detrimental bacteria in the biofilm in the oral cavity, without the use of abrasive or desiccating cleaning reagents. It should be noted the swab taken at the start of the trial indicated the level of detrimental bacteria present in the oral cavity during the patients previous oral care regime (i.e. their normal or standard care regime—not the method of the invention). The results show a significant improvement over the previous oral care regime.

1. A method of improving the health of the oral cavity, comprising the steps in any order of:
a) introducing into said oral cavity a liquid product containing at least:
about 5% to about 50% w/v of a saliva activating agent, an oxidising agent, and water,
thereafter removing said liquid product at least in part from the oral cavity,
b) introducing into said oral cavity, a product at least initially in powder form (“powder product”) containing at least the following ingredients,
about 30% to about 98% w/w of a saliva activating agent,
a pH adjusting agent, and
a remineralising agent,
thereafter agitating said powder product, whether dissolved or otherwise, in
said oral cavity at least in part with said oral tool, and thereafter removing the resultant product or products at least in part from the oral cavity.

2. The method of claim 1 wherein said saliva activating agent in said liquid product is present in a range of about 5% to about 30% w/v.

3. (canceled)

4. The method of claim 1 wherein said saliva activating agent in said liquid product is any one or more of:
non-acidic;
non-fermentable;
a caries reducing agent;
a plaque adhesion reducing agent.

5.-7. (canceled)

8. The method of claim 1 wherein said saliva activating agent in said liquid product is xylitol.

9. The method of claim 1 wherein said oxidising agent in said liquid product is present in a range of about 200 PPM to about 1000 PPM in solution.

10. (canceled)

11. The method of claim 1 wherein said oxidising agent has any one or more of the following properties:
a whitening and/or bleaching effect on teeth in said oral cavity;
at least temporarily reduces or temporarily substantially alleviates anaerobic bacteria in said oral cavity;
at least partially oxidises volatile sulphur compounds in said oral cavity, reducing instance of malodorous breath.

12.-13. (canceled)

14. The method of claim 1 wherein said oxidising agent is selected from anyone or more of the following:
stabilised chlorine dioxide,
hydrogen peroxide,
zinc chloride.

15.-16. (canceled)

17. The method of claim 1 wherein said liquid product has a pH of about 7 to about 9.

18.-19. (canceled)

20. The method of claim 1 wherein said liquid product is non-desiccating when introduced into said oral cavity.

21. The method of claim 1 wherein said saliva activating agent in said powder product is present in a range of about 30% to about 80% w/w.

22. (canceled)

23. The method of claim 1 wherein said saliva activating agent in said powder product is xylitol.

24. The method of claim 1 wherein said pH adjusting agent in said powder product is present in a range of about 1% to about 50% by weight.

25. (canceled)

26. The method of claim 1 wherein said pH adjusting agent is sodium bicarbonate.

27. The method of claim 1 wherein said remineralising agent in said powder product is present in a range of about 0.1% to about 30% by weight.

28. (canceled)

29. The method of claim 1 wherein said remineralising agent is selected from anyone of more of the following:
calcium glycerophosphate,
Calcium fluoride.

30. (canceled)

31. The method of claim 1 wherein said powder product substantially dissolves when introduced into said oral cavity.

32.-38. (canceled)

39. The method of claim 1 wherein the steps in the method are carried out in the order of step a) followed by step b).

40. (canceled)

41. The method of claim 1 wherein said method includes, in addition to and after steps a) and b) introducing into said oral cavity a lozenge product containing at least the following ingredients,
about 30% to about 95% by weight of a saliva activating agent, and a remineralising agent.

42.-44. (canceled)

45. A product for improving the health of the oral cavity, comprising,
about 30% to about 98% w/w of a saliva activating agent,
a pH adjusting agent, and
a remineralising agent,
wherein said product has a pH of about 7.8 to about 8.5, and
wherein said product is in solid form and wherein said product substantially dissolves when introduced into said oral cavity.

46. A kit of parts for improving the health of the oral cavity, comprising,
a powder product, and
a liquid product,
wherein said liquid product contains at least:
about 5% to about 50% w/v of a saliva activating agent,
an oxidising agent, and
water,
and said powder product contains at least:
about 30% to about 98% w/w of a saliva activating agent,
a pH adjusting agent,
a remineralising agent.

47.-61. (canceled)
